(19) United States
(12) Patent Application Publication
(10) Pub. No.: US 2009/0023999 A1
(43) Pub. Date: Jan. 22, 2009

(54) Miniature optical head with integrated scanning for producing a homogeneous image and confocal imaging system using said head

(75) Inventors: Gilles Mathieu, Lunel (FR); Magalie Genet, Guyancourt (FR); Bertrand Viellerobe, Nogent Sur Marine (FR); Frederic Berier, Plouarzel (FR)

Correspondence Address: YOUNG & THOMPSON, 209 Madison Street, Suite 500, ALEXANDRIA, VA, 22314, US

(73) Assignee: Mauna Kea Technologies, Paris (FR)

(21) Appl. No.: 10/584,854

(22) PCT Filed: Dec. 29, 2004
(86) PCT No.: PCT/FR2004/003402

§ 371 Date: Jun. 28, 2006

(30) Foreign Application Priority Data
Dec. 31, 2003 (FR) 0315627

(51) Int. Cl. A61B 1/06

(52) U.S. Cl. 600/160

(57) ABSTRACT

A miniature confocal optical head (4) for a confocal imaging system, in particular endoscopic, includes a point source (2a) for producing a light beam (13); a ball lens (12) arranged at the tip of the optical head, partly outside, to cause the light beam to converge in an excitation point (19) located in a subsurface field under observation (14) of a sample (15), the digital aperture of the lens and the dimension of the point source being adapted to ensure confocality of the assembly; and a scanner (10, 211, 22) for rotating the point source so that the excitation point (19) scans the field under observation. The system produces a real-time confocal image (about 10 images/sec.) of very high quality and homogeneous in the entire field (the optical aberrations are constant in the entire field due to the spherical symmetry of the ball lens), achieved through a miniature head.

This invention relates to an miniature optical head with integrated scanning for producing a homogeneous confocal image, as well as a confocal imaging system using this optical head.

It has a particularly useful application in the field of high resolution confocal imaging, making it possible to observe and analyze a biological tissue in situ in vivo, in particular via the operating channel of an endoscope (internal diameter comprised between 2 and 3 mm) or with an optical head integrated into the endoscope. The invention can also be applied to the fields of dermatology or gynaecology requiring less extreme miniaturization of the optical head, or also to the field of in situ biological analyses, on humans or small animals.

According to a first type of system, in particular described in the Patent Application WO 00/16151, an image guide is used, constituted by a bundle of flexible optical fibres, comprising at its distal end an optical focusing head intended to come into contact with the sample to be analyzed. The excitation beam scanning means are situated at the proximal end of the image guide provided for scanning the fibres in turn. The confocal character resides here, in particular, in the fact that the same optical fibre of the guide is used to convey the excitation signal and the return signal emitted. This type of system has the advantage of an optical head simplified from the mechanical point of view and thus essentially comprising optical focusing means which can be miniaturized. On the other hand, it has certain drawbacks, linked to the use of an array of optical fibres, in particular, the problem of sampling the tissue (continuity between the excitation points corresponding to the illumination of a fibre), the problem of injecting the fibres one by one and of the parasitic reflections at the inlet and outlet of the image guide in particular as regards backscattering, the sophisticated data processing of the image necessary in order to then correct the pattern of the fibres on the image, etc.

According to another known type of system, the beam scanning means are situated in the optical head at the distal end of a single flexible optical fibre. The confocal character is obtained here due to the fact that the optical fibre is used for conveying the excitation signal and return signal emitted with an appropriate core diameter of the fibre and numerical aperture.

The drawbacks of this type of system are then essentially linked to difficulties of miniaturizing the head, reproducibility and reliability of the mechanical means used for carrying out the scanning of the emergent beam of the optical fibre.

The document U.S. Pat. No. 6,091,067 describes a scanning system in which an optical fibre is fixed to two bimorphic piezoelectric shims, one of the shims is placed in the axial direction of the fibre and the other in the direction perpendicular to the optical axis. The shims, in order to offer an appropriate displacement relative to the field of view, must have a given length. Perpendicular to the axis of the optical fibre, this length constraint in fact leads to an optical head diameter too large for the in vivo applications in situ envisaged according to the present invention.

Several documents describe miniature confocal optical heads using micro-mechanical-type micro-mirrors (MEMs).

The Patent Application US 2002/0018276 describes a miniature confocal system using an optical fibre. The light leaving the fibre is reflected on the metallized part of a lens. This light is then reflected on a two-dimensional MEMs micro-mirror surrounding the fibre. The light is then sent towards the sample via an optical system. The light returning from the sample follows the reverse path and returns by the fibre which serves for spatial filtering. The system is miniature, being 2 mm in diameter and 2.5 mm in length.

The U.S. Pat. No. 6,154,305, U.S. Pat. No. 6,088,145, U.S. Pat. No. 6,007,208, U.S. Pat. No. 5,907,425, U.S. Pat. No. 5,742,419, U.S. Pat. No. 6,172,789 and U.S. Pat. No. 6,057,952 describe a confocal head in which the scanning of the field of view is carried out by two electrostatically pivoted MEMS micro-mirrors. The proposed head can be miniaturized but on the other hand offers a 60×60 μm field of view which is too small with respect to the applications according to the invention corresponding to a field measuring 100×100 μm minimum in order to be able to observe, for example, several cell nuclei which are 5 μm in diameter generally spaced out at intervals of several tens of μm. The number of images per second of 5 to 8 is moreover also insufficient for imaging in real time (requiring a minimum of 10 to 12 images per second in the slowest mode with 640 lines). Moreover also, for certain of them, the field of view is situated parallel to the axis of the optical fibre, which can lead to practical difficulties of use (correct positioning of the probe).

Generally, the change in direction of the optical beam by successive reflections on micro-mirrors leads to optical aberrations, in particular distortion or field curvature, which it is necessary to correct.

The document U.S. Pat. No. 6,294,775 discloses a miniature endoscopic system using an optical fibre which is made to resonate along two axes. A lens makes it possible to focus the beam leaving the fibre in the sample. The scanning has the characteristic of being carried out in a spiral. However, the images obtained have an inhomogeneous quality throughout the field of view.

The purpose of this invention is to remedy the above-mentioned drawbacks by proposing a system making it possible to obtain homogeneous images throughout the field.

Another purpose of the invention is to propose a head sufficiently miniature to be integrated in the operating channel of an endoscope for example.

Another purpose of the invention is a system capable of producing an image in real time (at least 10 images per second) and covering a field to be imaged of the order of 100 μm×100 μm minimum and preferably 150 μm×150 μm.

At least one of the above-mentioned purposes is achieved with a miniature confocal optical head for a confocal imaging system, in particular endoscopic, said head comprising a point source for producing a light beam. According to the invention, said optical head also comprises:
a ball lens arranged at the end of the optical head, preferably partially outside, in order to cause the light beam to converge into an excitation point situated in a subsurface field under observation of a sample, the numerical aperture of this lens and the specifications (diameter and numerical aperture) of the point source being suitable to ensure the confocality of the assembly, and
scanning means for displacing the point source in rotation so that the excitation point scans said field under observation.

The scanning consists of rotational movements along two axes passing through the centre of the ball lens.

The ball lens makes it possible to focus the laser beam inside the sample. It has numerous advantages:
a spherical symmetry: this symmetry associated with scanning in rotation makes it possible to obtain a homogeneous image since the aberrations remain constant throughout the field, unlike most of the devices of the prior art,
large numerical aperture (NA=1 in air): this large numerical aperture makes it possible to collect a maximum of photons originating from the focussing plane, and associated with the small diameter of the point source, it makes it possible to ensure a good confocality for the whole system,
small diameter: the diameter of the ball lenses can vary from a few tens of micrometres to a few tens of millimetres, this diameter which will determine the dimensions of the system must be chosen as a function of the size of the field under observation and of the site which is to be studied in order to confer a non-invasive character on it, and
ease of assembly: no problem of tilting with a ball lens which must be placed in a cylindrical head.

With the optical head according to the invention, sufficient miniaturization is achieved. In fact, the use of a ball lens and a point source, from a single optical fibre for example, makes it possible to reduce the space requirement of the system, and therefore the total diameter while retaining a large numerical aperture on the sample and a very good confocality criterion.

The confocal character and the homogenization of the aberrations in the field are necessary in order to obtain a good-quality image which exhibits no differences between centre and edge of the field linked to the optical device.

According to a first variant of the invention, during the scanning, the point source pivots independently of the ball lens. In this case, the distance between the point source and the centre of the ball lens is kept constant such that the field under observation is curved. The scanning means therefore act directly on the point source by moving it according to rotational movements around a hemisphere of the ball lens. The latter can advantageously remain fixed.

According to a second variant of the invention, during the scanning, the point source is integral with the ball lens. The latter can therefore pivot along two axes passing through its centre. Advantageously, the scanning means can act directly on the ball lens. An action can also be provided on the ball lens and on the point source. In this particular scanning mode case where the ball lens pivots, this ball lens must slide over the sample as it moves relative to the latter in order to produce the image. This therefore assumes that the sample remains fixed relative to the head and more particularly relative to the ball lens. In order to do this, the optical head according to the invention can advantageously comprise means for introducing a liquid between the external surface of the ball lens and the sample so as to facilitate the sliding of the ball lens over the sample. This liquid can for example consist of a film of water introduced via the optical head or formed naturally. Otherwise it is possible to provide a fine rigid curved plate used as a window designed to allow the ball lens to slide over the sample.

Preferably, the optical head can also comprise corrective optical means integral with the point source and arranged between this point source and the ball lens in order to correct residual aberrations of the ball lens.

Thus, according to the first scanning mode where the ball lens can remain fixed, the point source is integral with the corrective optics and both pivot along two rotational axes θ and φ relative to the ball lens. The distance between the corrective optics and the ball lens is kept constant over time. According to the second scanning mode, the assembly comprising point source+corrective optics+ball lens pivots along two rotational axes θ and φ with the centre of the ball lens as the centre of rotation. In this case, the corrective optics and the ball lens can be bonded.

In these two particular cases, the scanning means advantageously comprise means for carrying out scanning processes along the two rotational axes of the ball lens θ and φ so as to obtain a two-dimensional image in real time. Then scanning is carried out at a frequency of approximately 4 kHz in one direction in order to ensure a rate of 10 to 12 images/s.

Moreover, the scanning means are suitable for supporting the movement of the point source and of one or more optical systems (corrective optics alone or corrective optics+ball lens). The micro-mechanical scanning can be carried out by means of micro-motors, piezoelectric systems, or MEMs with any type of actuation which can be envisaged: electrostatic, magnetic, thermal etc. These scanning means are adjusted with precision in order to carry out a scan in a hemispherical plane which preferably perfectly follows the surface of the ball lens. The imaged field is thus curved.

According to a preferred embodiment of the invention, the optical head comprises the terminal part of an optical fibre suitable for conducting the light beam from an external source, the light beam emerging from the fibre constituting the point source. The optical fibre is preferably monomode with a core diameter and a numerical aperture allowing spatial filtering of the return signal and thus ensuring the confocality of the head.

In other words, the optical fibre is longitudinal monomode in order both to allow the illumination of the sample to be as homogeneous as possible and the spatial filtering on return to be the best possible. The numerical aperture of the optical fibre can be variable and chosen as a function of the desired magnification which is to be given to the device in order to optimize the excitation flux.

According to a variant, the point source is constituted by a VCSEL-type laser source, having a numerical aperture and a cavity outlet diameter compatible with a confocal system, and associated with a detector placed behind the VCSEL cavity.

According to another aspect of the invention, a confocal imaging system is proposed, comprising:
a confocal optical head with integrated scanning as defined above;
a source suitable for emitting a light beam;
means of detection of an emitted signal;
electronic and computer control and processing means of the signal emitted suitable for reconstructing a confocal image of an imaged field.

The system can also comprise an optical fibre connected to a laser source and coupling means for coupling said fibre with the optical fibre for transport to and from the optical head and a fibre for transporting the emitted signal to the detection means.

According to the invention, when the optical head comprises a VCSEL laser source and integrated detector, the system comprises flexible connecting means between the optical head and the signal processing means.

Other advantages and characteristics of the invention will become apparent on examination of the detailed description of a method of implementation which is in no way limitative, and of the attached drawings, in which:

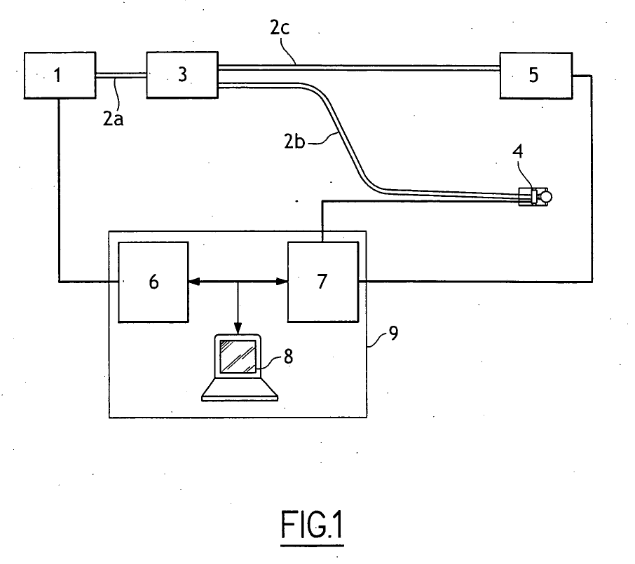

FIG. 1 is a general diagram of an example of a fibre-type confocal imaging system using the miniature head according to the invention;

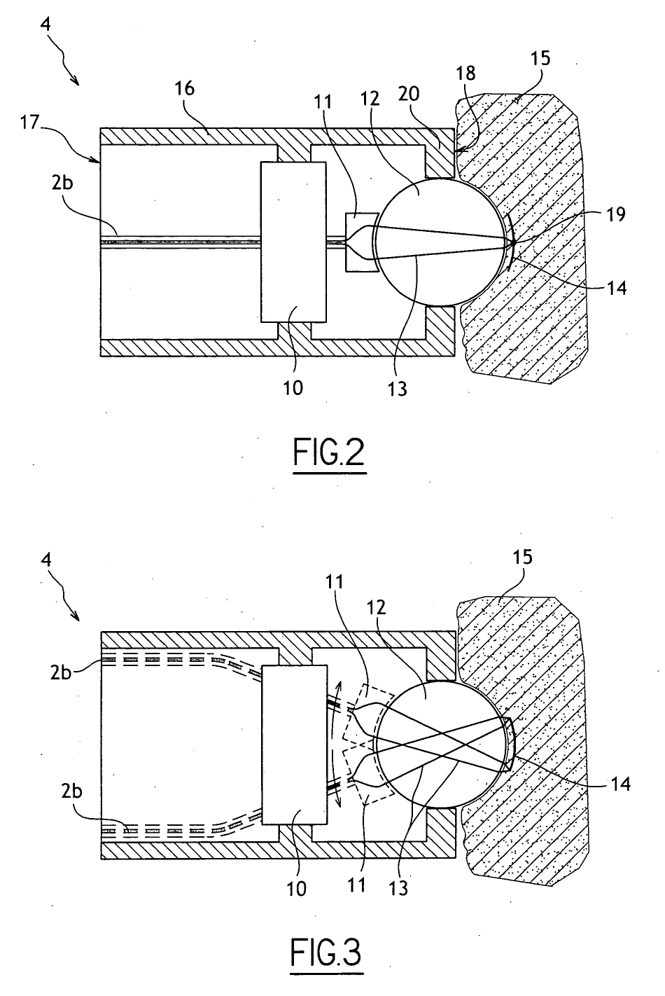

FIG. 2 is a side cross-section of a miniature head according to a first embodiment;

FIG. 3 is a cross-section of the miniature head of FIG. 2 for two distinct scanning positions;

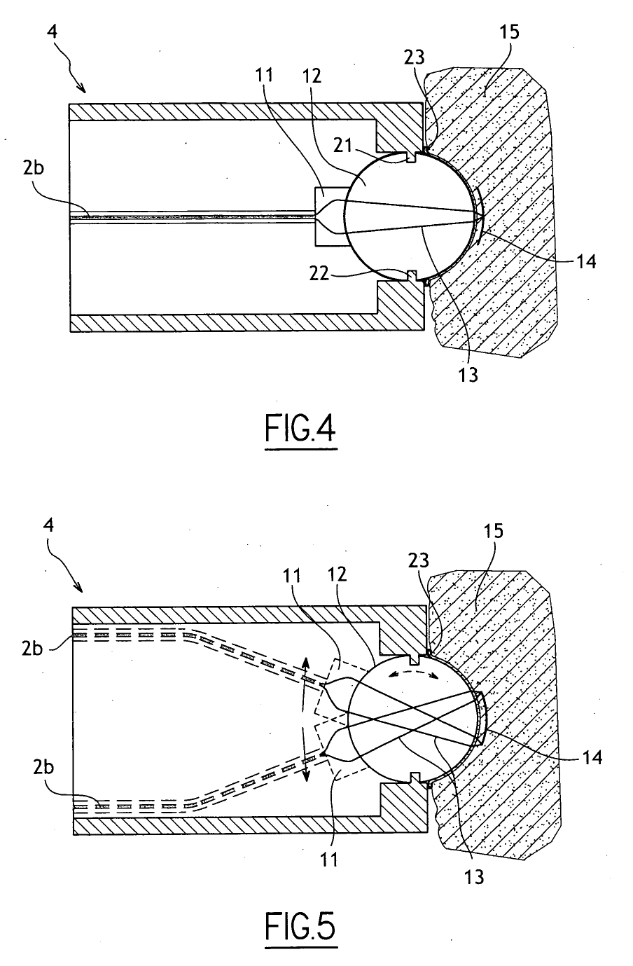

FIG. 4 is a side cross-section of a miniature head according to a second embodiment; and

FIG. 5 is a cross-section of the miniature head of FIG. 4 for two distinct scanning positions.

FIG. 1 diagrammatically represents a fibre-type confocal imaging system which can include a miniature head according to the invention.

The system comprises a source 1, for example a laser source, capable of emitting an excitation signal at a wavelength capable of generating in a sample a fluorescence or backscattering return signal, said signal being transported by a first monomode optical fibre 2a to coupling means 3, for example a 50/50 fibre coupler, provided in order to direct the excitation signal originating from the source 1 into a monomode optical fibre 2b at the end of which the miniature optical head 4 according to the invention is situated and in order to direct the return signal originating from the excited site towards detection means 5, for example a photodetector, using a third monomode optical fibre 2c. The system comprises a complete electronic and computer control unit 9 equipped with electronic control, command and synchronization means 6, making it possible to control the source 1, the scanning means of the optical head 4 and the detection means 5, in a synchronized manner, in order in particular to know the location of the signal in the sample in order to allow the construction of an image in real time. The unit 9 also comprises electronic means 7 of amplification, forming and A/D conversion of the signal detected by the detection means 5, data-processing means 8 comprising an acquisition card, a graphics card and means of displaying the images obtained.

This system operates overall in the following manner: the miniature optical head 4 is brought into contact with a sample to be analyzed, for example via the operating channel of an endoscope. The source 1 sends an excitation signal or laser beam with a chosen wavelength into the portion of fibre 2a. The coupler 3 directs the excitation signal into the portion of fibre 2b guiding the signal into the optical head 4 where it is scanned and focused on an analysis surface (or analysis field) at a given depth in the sample. A return signal originating from the scanned surface in the sample follows the reverse path of the excitation signal as far as the coupler 3: it is collected by the optical means of the optical head 4, recoupled in the portion of fibre 2b, then directed by the coupler 3 into the portion of fibre 2c towards the detector 5. The signal detected is amplified and converted into a digital signal, then subjected to data-processing in order to constitute an image element displayed in real time.

The miniature head according to the invention is now described in detail with reference to the chosen embodiments represented in FIGS. 2 to 5.

FIGS. 2 and 3 show a first embodiment of the optical head 4 according to the invention. This head 4 is a mechanical support structure constituted by a hollow body 16, for example a tubular optics holder open at a first end 17 and tightly closed at a second end 18. The optical fibre 2b penetrates as far as the head 4 via the opening 17. The end of the optical fibre 2b is integral with a corrective optics 11.

In the axis of the laser beam 13 leaving the optical fibre 2b, after the corrective optics 11, a ball lens 12 is situated making it possible to focus this laser beam into an excitation point, the spot 19, situated in the sample 15, which is for example the tissue of a living organism. The corrective optics and the ball lens make it possible to focus the light at a depth of a few tens of microns in the sample.

The light originating from the tissue 15 returns into the optical head assembly before being re-coupled in the optical fibre 2b which serves for spatial filtering. The confocal character of the device which consists of detecting only the photons originating from this depth is ensured by the characteristics of the optical fibre 2b and the corrective optics assembly 11 and ball lens 12.

The optical fibre is longitudinal monomode in order to allow both the illumination of the tissue 15 to be as homogeneous as possible and the spatial filtering on return to be the best possible. The numerical aperture of the fibre is chosen in order to allow an optimized collection of photons, and to allow, together with an appropriate core diameter, a coupling of the return signal in the optical fibre 2b, and therefore spatial filtering which is the best possible. Typically, the numerical aperture varies between 0.2 and 0.4 as a function of the magnification which is to be given to the system, and the core diameter is comprised between 1 and 2 μm.

The corrective optics 11, placed between the optical fibre 2b and the ball lens 12, in particular has the function of correcting residual aberrations of the ball lens 12 and optionally minimizing the aberrations linked to the scanning. It can be constituted by one or more refractive (doublets, triplets, index gradient lenses etc.) or diffractive lenses. The number of lenses is relatively limited such that the weight moved during the scanning is low. According to the embodiment of FIGS. 2 and 3, the corrective optics 11 are integral with the optical fibre 2b but not with the ball lens 12.

The ball lens is tightly shimmed into a circular orifice produced in the exit window 20 of the optical head. This ball lens 12 is partially arranged outside the body 16 such that when the head 4 is positioned on the tissue 15, the outer part of the ball lens constitutes a protuberance pushing into the tissue 15.

In order to produce a two-dimensional image of the field under observation 14, the laser beam 13 scans so that the spot 19 describes this field under observation 14. To do this, the optical head 4 comprises scanning means 10 supported by the body 16 and arranged so as to move the optical fibre 2b-corrective optics 11 assembly in a hemispherical plane. The field under observation 14 is then a hemispherical or more generally curved field. The corrective optics 12 have a face which moulds to the shape of the ball lens 12 without ever coming into contact with it. The interspace between the corrective optics 11 and the ball lens 12 remains constant during the scanning. This scanning is carried out along two axes passing through the centre of the ball lens. FIG. 3 shows two extreme positions of scanning along an axis. The pivoting angle is chosen so as to allow an field under observation 14 of large dimensions and the scanning speeds are such that the images are obtained in real time (at least 10 images per second). The scanning system can comprise piezoelectric means (not shown) and MEMs means (not shown) for respectively carrying out a rapid scanning along a first axis at a frequency of approximately 4 kHz and a slow scanning along a second axis perpendicular to the first at a frequency between 10 and 15 Hz.

All of the elements included in the optical head 4 have dimensions compatible with a miniaturization of the head which must have a total external diameter of 2 to 3 mm maximum. The elements actuated by the scanning means must be resistant and capable of responding to mechanical constraints.

In the embodiment of FIGS. 2 and 3, the ball lens 12 is no longer integral with the optical fibre 2b and can thus remain fixed. The second embodiment, represented in FIGS. 4 and 5, on the other hand consists of an integral assembly comprising the optical fibre 2b, corrective optics 11 and ball lens 12. The interspace between the corrective optics 11 and the ball lens 12 is eliminated. The second mode also differs from the first mode of FIGS. 2 and 3 in that the scanning means 21 and 22 are associated with the exit window 20 and cause the ball lens 12 to pivot along two rotational axes passing through the centre of the ball lens 12.

In this case of direct scanning on the ball lens 12, a film of water 23 is formed on the external face of the ball lens to ease the sliding over the outer surface of the tissue 15. The water can be introduced by a pipe (not shown) via the optical head 4, but it can also be formed by other means. A window as defined previously can also be used instead or in combination.

Three possible examples of dimensioning of the miniature laser scanning head according to the invention are now cited below:

### EXAMPLE 1

Field source: 500 μm×500 μm
Scanning angle (θ, φ): +/−3.7°
Numerical aperture of the optical fibre 2b: ON=0.25
Core diameter of the optical fibre 2b: Ø_{core}=2.1 μm
Diameter of the ball lens 12: Ø_{L}=2 mm
Fibre end—ball lens centre distance≦3.8 mm
Numerical aperture of the ball lens: NA_{L}=1.25 in water
Magnification of the optical system: M=5
Imaged field 14 in the tissue 15: 100 μm×100 μm
Diameter of the spot 19 focused in the tissue: limited by the diffraction throughout the field

### EXAMPLE 2

Field source: 500 μm×500 μm
Scanning angle (θ, φ): +/−6.22°
Numerical aperture of the optical fibre 2b: NA=0.4
Core diameter of the optical fibre 2b: Ø_{core}=1.31 μm
Diameter of the ball lens 12: Ø_{L}=2 mm
Fibre end—ball lens centre distance≦2.29 mm
Numerical aperture of the ball lens: NA_{L}=1.20 in water
Magnification of the optical system: M=3
Imaged field 14 in the tissue 15: 166 μm×166 μm
Diameter of the spot 19 focused in the tissue: limited by the diffraction throughout the field

### EXAMPLE 3

Field source: 300 μm×300 μm
Scanning angle (θ, φ): +/−7.5°
Numerical aperture of the optical fibre 2b: NA=0.4
Core diameter of the optical fibre 2b: Ø_{core}=1.31 μm
Diameter of the ball lens 12: Ø_{L}=1 mm
Fibre end—ball lens centre distance≦1.14 mm
Numerical aperture of the ball lens: NA_{L}=1.20 in water
Magnification of the optical system: M=3
Imaged field 14 in the tissue 15: 100 μm×100 μm
Diameter of the spot 19 focused in the tissue: limited by the diffraction throughout the field.

Example 1 compared with Example 2 has a greater magnification and as a result better lateral and axial resolution, but to the detriment of the field of view. Example 3 compared with Example 2 possesses a smaller field image, but requires less space (Ø=1 mm instead of 2 mm) and has better accessibility as a result. Another advantage of the invention is that, in the two scanning cases described previously, the scanning amplitude is low, therefore a design easier to produce. As a result, the dimensioning of the system must be adapted to the object of study, to the field of application and to the operating mode which can be either a fluorescence imaging mode or a backscatter imaging mode.

The system according to the invention makes it possible to obtain a very good-quality confocal image in real time (approximately 10 images/s) which is homogeneous throughout the field, by means of a miniature laser scanning head (diameter of a few mm). Such a configuration must make it possible to image the sites which are difficult to access in vivo on humans or animals without being invasive (endoscopic case) or being only very slightly invasive (case of micro-incisions).

Of course, the invention is not limited to the examples which have just been described and numerous variations can be applied to these examples without going beyond the scope of the invention. In fact, when the point source (optical fibre, VCSEL), the corrective optics and the ball lens are integral, it is possible to envisage scanning modes according to which the scanning means are in direct connection with the point source (first mode) and/or with the corrective optic.

1. Miniature confocal optical head (4) for a confocal imaging system, in particular endoscopic, said head (4) comprising a point source for producing a light beam (13), characterized in that it also comprises:
a ball lens (12) arranged at the end of the optical head (4), in order to cause said light beam (13) to converge into an excitation point (19) situated in a subsurface field under observation (14) of a sample (15), the numerical aperture of this lens and the dimensions of the point source being suitable to ensure the confocality of the assembly, and
scanning means (10, 21, 22) for displacing the point source in rotation so that the excitation point (19) scans said field under observation.

2. Optical head according to claim 1, characterized in that, during scanning, the point source pivots independently of the ball lens.

3. Optical head according to claim 2, characterized in that, during scanning, the distance between the point source and the centre of the ball lens is kept constant so that the field under observation (14) is curved.

4. Optical head according to claim 1, characterized in that, during scanning, the point source is integral with the ball lens.

5. Optical head according to claim 4, characterized in that it also comprises means for introducing a liquid (23) between the external surface of the ball lens and the sample so as to ease the sliding of the ball lens over the sample.

6. Optical head according to claim 4, characterized in that it also comprises a fine rigid curved plate used as a window designed to allow the ball lens to slide over the sample.

7. Optical head according to claim 4, characterized in that the scanning means (21, 22) act directly on the ball lens.

8. Optical head according to claim 1, characterized in that the scanning means (10) act directly on the point source.

9. Optical head according to claim 1, characterized in that it also comprises corrective optical means (11) integral with the point source and arranged between this point source and the ball lens (12) in order to correct residual aberrations of the ball lens.

10. Optical head according to claim 1, characterized in that the scanning means comprise means for carrying out scanning along two rotational axes of the ball lens so as to obtain a two-dimensional image in real time.

11. Optical head according to claim 10, characterized in that the scanning along one of the rotational axes reaches a frequency of approximately 4 kHz.

12. Optical head according to claim 1, characterized in that the scanning means comprise micro-motors.

13. Optical head according to claim 1, characterized in that the scanning means comprise piezoelectric elements.

14. Optical head according to claim 1, characterized in that the scanning means comprise MEMs-type micromechanical means.

15. Optical head according to claim 1, characterized in that it comprises the terminal part of an optical fibre suitable for conducting the light beam from an external source, the light beam emerging from the fibre constituting the point source.

16. Optical head according to claim 15, characterized in that the optical fibre is monomode with a core diameter and a numerical aperture allowing a spatial filtering of the return signal and therefore ensuring the confocality of the head.

17. Optical head according to claim 1, characterized in that the point source is constituted by a VCSEL-type laser source, having a numerical aperture and a cavity outlet diameter compatible with a confocal system, and associated with a detector placed behind the VCSEL cavity.

18. Confocal imaging system comprising:
a confocal optical head (4) with integrated scanning;
a source (1, 2a, 2b) suitable for emitting a light beam;
means of detection (5) of an emitted signal;
means (9) for electronic and computer control and processing of the signal emitted suitable for reconstructing a confocal image of a field image,
characterized in that the optical head (4) is according to claim 1.

19. System according to claim 18, characterized by an optical fibre (2a) connected to a laser source (1) and coupling means (3) for coupling said fibre (2a) with the optical fibre (2b) for transport to and from the optical head (4) and a fibre (2c) for transporting the emitted signal to the detection means.

20. System according to claim 18, characterized in that, the optical head comprising a VCSEL laser source and an integrated detector, the system comprises flexible connection means between the optical head and the signal processing means.
